(19) United States
(12) Patent Application Publication
(10) Pub. No.: US 2008/0275339 A1
(43) Pub. Date: Nov. 6, 2008

(54) DETERMINATION OF SOUND PROPAGATION SPEED IN NAVIGATED SURGERIES

(76) Inventors: Ingmar Thiemann, Munchen (DE); Fritz Vollmer, Munchen (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 12/113,573

(22) Filed: May 1, 2008

Related U.S. Application Data
(60) Provisional application No. 60/917,143, filed on May 10, 2007.

(30) Foreign Application Priority Data
May 3, 2007 (EP) 07107418

(51) Int. Cl. A61B 8/00

(52) U.S. Cl. 600/437

(57) ABSTRACT

The patent discloses a method and device for measuring the speed of sound of ultrasound waves used in a sonographic examination of a patient's body structure. Marker devices are attached to an ultrasound probe and to an ultrasound reflecting body structure such that the distance traveled by the ultrasound waves can be determined by a navigation system. Using this distance and the transit time of the ultrasound waves, the measured speed can be calculated. The calculated speed of sound may then be used to accurately scale the ultrasound images.

### RELATED APPLICATION DATA

This application claims priority of U.S. Provisional Application No. 60/917,143 filed on May 10, 2007, and EP 07107418 filed on May 3, 2007, which are incorporated herein by reference in their entirety.

### FIELD OF THE INVENTION

The invention relates to determining the speed of ultrasound waves used for sonography (and echography) in an anatomical body structure in which propagation of the ultrasound waves occurs. The determined speed of the ultrasound waves may be used to increase the accuracy of an ultrasound examination conducted during a navigated surgery.

### BACKGROUND OF THE INVENTION

In sonography, evaluating the measurement data (for example, scaling an ultrasound image) is often based on the assumption that the speed of sound is constant when passing through all body structures. Because determination of distances using sonography relies on the speed of sound, an incorrect assumption concerning the speed of sound can lead to incorrect distances. In such cases, the scaling of sonographic images may be incorrect.

For information regarding determination of the speed of sound in sonography, see: U.S. Pat. Nos. 4,056,907; 4,781,199; 4,669,482; and 5,638,820 (all of which are incorporated by reference herein).

### SUMMARY OF THE INVENTION

A sonographic apparatus in accordance with the invention measures or determines the speed of ultrasound waves that are emitted and then detected after reflection. The sonographic apparatus can be designed “in one part” (for example, a combined transmitter and receiver). One such example is a conventional ultrasound probe that transmits and receives ultrasound waves. In such a sonographic apparatus, the location where the ultrasound waves leave the sonographic apparatus may correspond to the location where said ultrasound waves are received by the sonographic apparatus. Additionally, the two locations may have fixed and/or predetermined positions relative to each other. For example, the transmitter (e.g., ultrasound source) and the detector (e.g., ultrasound receiver) may lie next to each other in the head of an ultrasound probe.

Another example of a sonographic apparatus in accordance with the invention may be designed such that the transmission and reception locations do not correspond and the relative position of the two locations to each other is variable. Such a sonographic apparatus is referred to herein as a “two-part” sonographic apparatus. The two-part sonographic apparatus may include a first location for emitting the ultrasound waves in a first part of the sonographic apparatus (e.g., an ultrasound source) and may further include a second location, spatially separate from the first location, for receiving the ultrasound waves in a second part of the sonographic apparatus (e.g., an ultrasound receiver). The two parts can be moved relative to each other, such that the relative position between the two locations is variable. A body structure to be examined lies between the ultrasound transmitter (the first part) and the ultrasound receiver (the second part) of the two-part sonographic apparatus.

A sonographic apparatus referred to herein as the one-part sonographic apparatus generally operates by: emitting ultrasound waves that pass through a body structure; the waves are reflected; the waves pass back through the body structure; and the one-part sonographic apparatus detects the reflected waves. The waves can be reflected by a part of the body structure that reflects ultrasound (“reflection part”). One example of a refection part is a bone structure. In general, the wave reflection is caused by the change in propagation of the ultrasound wave when the ultrasound wave reaches the reflection part. In other words, reflection occurs at the boundary area between the upstream body structure and the reflection part. Reflection at the reflection part is defined and used herein to mean reflection at the boundary area. Thus, reflection occurs when the ultrasound wave reaches a transition between two different materials (having different ultrasound propagation properties). One example is a transition from tissue to bone. A body structure that absorbs rather than reflects ultrasound waves, such as air-filled hollow spaces, are seldom useful as reflection parts.

Reflection can be caused by a bone structure or an exogenous part. For example, a plate that reflects ultrasound can be arranged on the side of the body structure opposing the ultrasound probe. The terms “one-part” and “two-part” above do not exclude the possibility of the sonographic apparatus comprising more than two parts, in addition to the transmitter and receiver.

A method in accordance with the invention may include the following. Data can be provided relating to reflection of the ultrasound waves (“reflection data”). The reflection data should contain information concerning the position of the reflection part relative to a reflection marker device. The reflection marker device is a marker device that has a fixed spatial relationship to the reflection part. For example, it may be rigidly connected to the reflection part. The marker device may be active or passive. For example, the device may actively emit or passively reflect energy. The energy used can include infrared light or sound waves. The marker device may include a plurality of reflective spheres that are fixed in a spatial relationship to each other. In one example, reflection data may be obtained during patient registration.

Data may also be provided relating to the location of emitting and receiving the ultrasound waves (“location data”). The location data describes the position of a sonographic apparatus marker device relative to an initial location and a receiving location of the ultrasound waves. A sonographic apparatus marker device may be provided in a fixed position relative to the sonographic apparatus. The sonographic apparatus marker device is a marker device similar to the marker device described above and may be rigidly connected to the sonographic apparatus. In other words, the relative position between the emitting location of the ultrasound waves relative to the sonographic apparatus marker device should be fixed. Additionally, the relative position between the receiving location of the ultrasound waves and the sonographic apparatus marker device should also be fixed. In one example, location data may be obtained during instrument calibration.

Data may also be provided relating to the relative locations of the marker devices. This data is referred to herein as “marker data.” The marker data describes the relative positions of the sonographic apparatus marker device and the reflection marker device.

Data may also be provided relating to transit time. This data is referred to as “transit time data.” The transit time data describes the transit time of an ultrasound wave propagating from an initial location to a receiving location.

For use herein, the path of the ultrasound wave from the initial location to the receiving location, and the corresponding length of said path, is the path from the initial location to the location where the ultrasound wave is reflected and from that location to the receiving location. The path length traveled by the ultrasound wave may be calculated using the reflection data, the location data, and the marker data.

If the path length is known, then the speed of sound of the ultrasound wave can be determined from the transit time described by the transit time data. This determination yields the mean speed of sound that the sound wave has during the transit time. If the body structure that is permeable to ultrasound is heterogeneous with regard to the speed of sound, then the speed of sound determined as above can deviate locally from the mean speed of sound.

The reflection part can comprise an expanded area, such that the ultrasound waves passing along the path can potentially be reflected at different locations on the reflection part. The reflection location may be determined from information concerning an area of the reflection part and information concerning the direction of propagation of the ultrasound waves from the initial location. The point of intersection between ultrasound waves propagating from the initial location in a known direction along a propagation line, and the area of the reflection part, represents the reflection location.

As an alternative to the above-mentioned determination of the reflection location or in addition to the determination, the refection location may also be determined for a given area of the reflection part in accordance with the principle that the angle of incidence equals the angle of reflection. This principle is useful if the initial location does not correspond to the receiving location.

The reflection data may be ascertained in several ways. If the reflection part is situated outside a patient's body, then the reflection data can be ascertained by scanning, sensing or optically detecting the reflection part, and determining the position of the reflection part relative to the reflection marker device. If the reflection part is situated in the interior of a patient's body, for example, a bone structure, then the reflection data may be determined using a medical analysis method that employs waves or beams to obtain scaled information concerning the position and/or spatial distribution of components of a body structure. Commonly available methods include x-ray analysis methods (for example, CT or x-ray recordings or fluoroscopic images, that may include 3D images reconstructed from said x-ray analysis method) or nuclear magnetic resonance recordings (for example, NMR, differential NMR, etc.). The analysis may be performed with a marker device attached to the patient's body. In such cases, the marker device is configured to be detected both by the analysis apparatus and by a marker detection device (for example, a camera or a sensor that responds to emitted or reflected energy). For example, one may use marker spheres that both reflect light and contain a metallic core that can be detected by the x-ray apparatus. The marker spheres may be rigidly connected to the bone structure. For example, a sphere-mounting device may be screwed into the bone structure. Using such marker spheres, it is possible to determine the relative position of the reflection marker device to a part of the body structure (for example, a bone) where the sound waves are potentially reflected. The information concerning the position of the area of the reflection part may include information concerning a profile of the area. The area (and its profile) can be calculated by a mathematical function to determine the point of intersection between the propagation line and the reflection area.

In the medical analysis for determining the position of a reflective body structure, an analysis method may be employed to determine the reflection data that does not involve sonography. Therefore, the analysis data obtained does not depend on the speed of sound of the ultrasound waves.

As stated above, the sonographic apparatus can be a two-part sonographic apparatus. In this case, the ultrasound waves ordinarily pass from the initial location through the body structure and then strike the receiving part of the sonographic apparatus. The distance between the initial location and the receiving location determines the path.

In the two-part sonographic apparatus, there are at least two separate marker devices for the sonographic apparatus. A receiver marker device is attached to the receiving part of the sonographic apparatus providing a fixed position relative to the receiving location of the ultrasound waves. A transmitter marker device is attached to the transmitting part of the sonographic apparatus providing a fixed position relative to the initial location of the ultrasound waves. By determining the positions of the transmitter marker device and the receiver marker device, and on the basis of the known relative position between the transmitter marker device and the initial location and the receiver marker device and the receiving location, it is possible to determine the distance between the initial location and the receiving location. The positions of the marker devices are determined using a sensor such as a camera. This process also determines the path length that the ultrasound waves travel.

Medical analysis methods, including those mentioned above, may be employed to obtain information concerning the body structure that is examined or is to be examined using sonography. These medical analysis methods may be used to identify different types of body structures in the case of a heterogeneous body structure and to determine their arrangement relative to a marker device (for example, a reflection marker device).

A database may be provided that assigns different speeds of sound of the ultrasound waves for propagation through different types of parts of the body structure (for example, blood or tissue). When the different types of body structure are identified and their positions and arrangement is determined using NMR, a corresponding speed of sound may be assigned to the individual types. It is then possible to determine a spatial distribution of the speed of ultrasound waves throughout the body structure. This spatial distribution may be calibrated by determining the speed of ultrasound waves in accordance with the invention. The calibration may be configured such that the mean speed of sound through the body structure is adapted to the speed of sound determined in accordance with the invention. This process may be performed such that a mean speed of sound (based on the database: a “database speed of sound”) is determined. This determination may be made along the sound path from the stored spatial distribution data and the medical analysis. The value determined may be compared with the speed of sound calculated (“measured mean speed of sound”) in accordance with the invention, that represents a mean value along the measurement path. The speed-of-sound values for the individual regions of the body structure that are stored in the database may be raised or lowered by a particular percentage, until the mean database speed of sound corresponds to the measured mean speed of sound. In this manner, a calibrated spatial distribution of the speed of sound in the body structure is obtained. This spatial distribution may be used to increase the accuracy in scaling ultrasound images.

Additional examples of different types of body structures may include healthy and diseased tissue, for example, healthy brain regions and a brain tumor. If the different speed of sound corresponding to the different types of body structure is taken into account and then calibrated in accordance with the method described above by measuring the mean speed of sound, it is possible to further increase the accuracy in scaling. In addition to the medical analysis methods mentioned above, sonography may be employed as a medical analysis method for typifying the body structure and measuring it in a spatial resolution and determining its position.

A device in accordance with the invention may also include a data processing device. The data processing device may be designed to process the data in the manner presented. The device may include an input device for inputting the data to be processed and also may include a sonographic apparatus that serves to ascertain the transit time data. Additionally, a detection device (for example, a camera) may be provided to detect marker device positions.

The device in accordance with the invention may also include a medical analysis apparatus (for example, an NMR apparatus or CT apparatus) wherein the measurement data may be transferred to the data processing device.

The device in accordance with the invention may also include a software program that, when it is executed on the data processing device, performs a method in accordance with the manner presented. The data to be processed by the software program may be provided to the data processing device via common input devices. Examples include a keyboard, interfaces to data memories (for example, a disc drive or an optical disc), or access to other databases stored at a remote location (for example, via the Internet).

### BRIEF DESCRIPTION OF THE DRAWINGS

The forgoing and other features of the invention are hereinafter discussed with reference to the figures.

FIG. 1 illustrates an exemplary configuration of a device in accordance with the invention used for examination of a patient's brain.

FIG. 2 illustrates the principle of measuring the speed of ultrasound waves using the exemplary configuration of FIG. 1.

FIG. 3 illustrates the measurement of the speed of ultrasound waves using a two-part sonographic apparatus.

FIG. 4 illustrates an exemplary configuration of a device in accordance with the invention used for examination of a patient's head.

FIG. 5 illustrates an exemplary data processing device or computer in accordance with the invention.

### DETAILED DESCRIPTION

Methods and devices in accordance with the present invention enable the mean speed of sound in a body structure (for example, in a live brain or in other body structures) to be determined. The body structures can be homogeneous or heterogeneous. In the case of heterogeneous body structures, a method and device are disclosed that utilize data from medical analysis device (see above). In the following description, examinations of both homogeneous and heterogeneous body structures are discussed.

Homogeneous tissue may be bordered by a different type of body structure that differs with regard to its ultrasound propagation properties. This border leads to reflection. For example, the tissue could be partly surrounded by a bone, as is the case with the brain.

During sonographic examination, the position of a sonographic apparatus (in particular, the positions of transmitting or receiving ultrasound waves, or the position of an ultrasound probe) may be monitored using a navigation apparatus. To this end, a marker device (for example, a reference star) may be attached to the transmitter, receiver, and/or ultrasound probe, such that the ultrasound probe can be navigated such as in IGS (image-guided surgery). It is possible to measure the position of the transmitter, receiver and/or ultrasound probe using a detection device that is a part of a navigation apparatus.

The ultrasound waves may be measured in the so-called A-mode or B-mode. A number of ultrasound scans may be performed to increase the accuracy of the measurement data. In addition to measuring the ultrasound waves, another medical analysis apparatus may be employed to determine reflection data and/or body structure data. For example, a CT or MRI may be used. The data obtained using the medical analysis apparatus may be analyzed. The analysis can be performed manually, for example by a physician, or automatically. The analysis may be directed to identifying types having different ultrasound propagation properties (for example, a different echogenicity), and may assist in determining the body structure data's position relative to markers and/or relative to homogeneous tissue that is permeable to ultrasound.

When a value for the speed “S” of the ultrasound waves is obtained, this value can be used to correct an ultrasound setting of the sonographic apparatus. In many situations, a sonographic apparatus (ultrasound probe) uses a fixed S value of 1540 meters/second. This value can be corrected in the above-mentioned manner to more accurately scale the ultrasound images and to be able to more accurately determine distances using ultrasound. With a corrected value, a greater accuracy in navigated sonography (ultrasound imaging technology) can be obtained.

In many situations, the speed of the ultrasound waves in a tissue is assumed, and the assumed speed corresponds to a mean value, such as is given for a healthy tissue or a tissue having a defined change (for example, adiposis hepatica). However, some illnesses cause a change in the speed of ultrasound (for example, by increasing the amount of water in the tissue, or, in the case of cysts or compressed tissue, there may exist hyper-dense tumors). The device and method in accordance with the present invention can measure the change in the speed of sound, even if changed due to a change in water content. Also, a database can be created using the data measured in this manner. For example, the database can be created with data for the speed of ultrasound that depends on the respective type of body structure and where the speed is specific to the respective type. This database can then be used, as explained above, to determine a spatial distribution of the speed of sound in the body structure being examined, for example, by determining the database speed of sound.

In particular, the device or method allows the speed of sound to be measured in-vivo, in the brain or in other body structures of a patient. As stated above, the value for the speed of sound that is generally fixed and predetermined in the prior art can be corrected to increase accuracy. In particular, this correction increases accuracy in the navigation of an instrument or implant and improves scaling and measuring lengths in ultrasound images.

Measuring and determining the speed of ultrasound in accordance with the invention also allows determination of the properties of the body structure. If a speed of sound is measured that deviates from healthy tissue, then it may be deduced that the tissue is diseased. Such deduction can allow identification of a tumor or changes in the speed-of-sound property of the tumor can be determined, so as to identify a possible malignant change in the tumor.

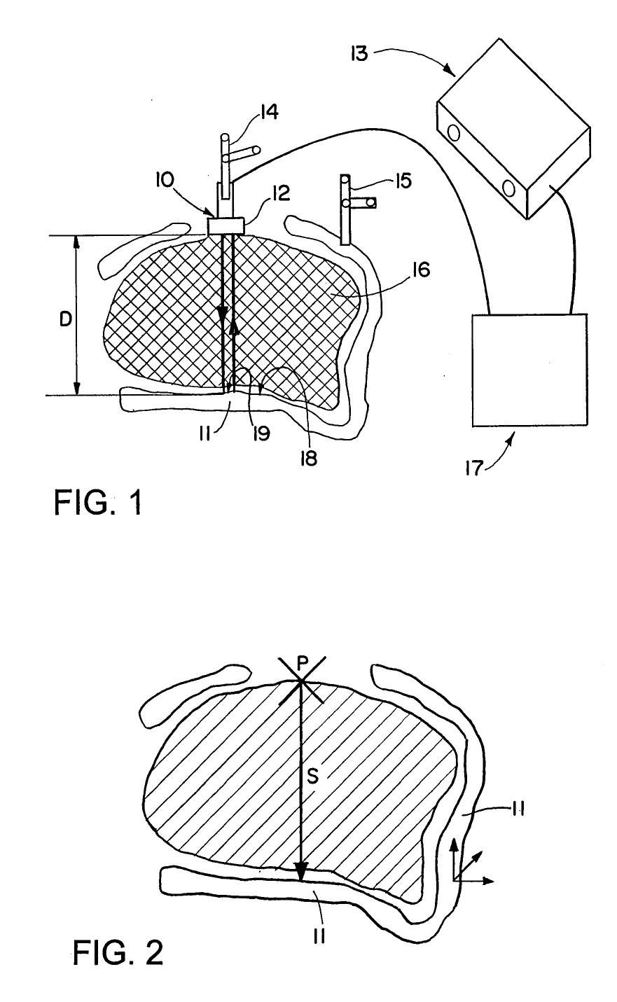

The principle of measuring the speed of the ultrasound waves is as follows. The mean speed of the ultrasound waves through a body structure is given by the path distance traveled by the ultrasound, divided by the time (transit time) that the waves require to travel the path distance. Turning to FIG. 1 for an example, the ultrasound waves require the time “T” to pass from an ultrasound probe 10 to a bone structure 11 (that represents an example of a reflection part). If the ultrasound waves are reflected by the bone structure 11 and measured by the same ultrasound probe 10, then for a distance “D” between the probe and the reflective bone structure, the speed of sound S is calculated as follows:

S=D/(T/2).

To determine the path length that the ultrasound travels from a head of the probe 12 to the bone structure 11, a navigation system 13 may be used to detect the position of marker devices 14 and 15. Marker device 14 is rigidly connected to ultrasound probe 10 and marker device 15 is rigidly connected to the bone structure 11. Data may additionally be used that contains the position of the bone structure 11 relative to the marker device 15. CT data may be used for this purpose, wherein the marker device 15 has been detected by the CT recording. “D” in FIG. 1 indicates the distance between the reflective part of the bone structure 11 and the head of the ultrasound probe 10. A black line with arrows indicates the ultrasound wave that passes forward and back between the head and the reflective part. The brain 16 is shown crosshatched.

Also shown in FIG. 1 is a data processing device 17 that may be operatively connected to the ultrasound probe 10 and/or the navigation system 13.

The position of a reflective part 18 of the bone structure 11 can be determined by a physician and transferred as input data into the data processing device 17. The position of the reflective part 18 can also be determined automatically by determining the direction of propagation of the ultrasound waves from the ultrasound probe 10. A reflection location 19 is the location at which a line running in this direction intersects with the bone structure 11. The relative position of the ultrasound probe 10 relative to the bone structure 11 may be determined by detecting the marker devices 14 and 15. In this manner, analysis data from a previously performed medical analysis method can be compared with the presently acquired ultrasound data. Additionally, bone structures may be identified automatically in the CT image by segmentation (for example, a structure recognition method).

At least some of the following information is provided to the data processing device 17 (or the conditions confirmed) to determine the speed of ultrasound waves using the medical analysis data (for example, CT data).
Registration information regarding the patient data (analysis data) may be provided in a reference frame (for example, the reference frame of a navigation system).
The medical analysis data (data not obtained using ultrasound) that enables the identification of the osseous body structure where reflection of ultrasound waves can occur. This identification can be automatic or manual (for example, by the physician).
The tissue between the ultrasound head and the reflection bone is preferably homogeneous
The tissue being examined using ultrasound is surrounded by a bone structure or at least partly borders a bone structure such that the ultrasound can be reflected back to the head of the probe.
The distance to the bone (or to another structure where ultrasound waves are reflected) can be reached by the ultrasound waves. For example, the ultrasound probe should be selected with regard to its output and frequency such that the waves arrive at the reflective structure, where they can be reflected.

The following steps may be performed before measuring the speed of the ultrasound waves:
The bone structures may be identified, in particular with regard to their position, and may be registered and/or spatially correlated with the ultrasound measurements. A region of the reflective structure where the reflection is to occur may be established before measuring the ultrasound waves.
The ultrasound probe may be detected or “calibrated.” In other words, the spatial relationship between the head of the ultrasound probe (for example, the exit point of the ultrasound waves) and the marker device (for example, a reference star) is determined. This registration may be performed in the reference frame of a navigation system.
The patient may be “registered” in the reference frame of the navigation system and/or device using marker device 15. In so doing, the non-ultrasound data set (analysis data) is registered relative to the ultrasound probe. The position of the previously identified reflective part is loaded into the device and/or determined using the analysis data.

The measurement for determining the speed of ultrasound may be performed in the so-called A-mode of the ultrasound probe. In doing so, a single sound pulse is emitted along a straight line, and the sound pulse reflected by the bone is received. The measurement can also be obtained in the so-called B-mode.

When the reflected pulse is received, the transit time T of the sound pulse wave from the ultrasound probe to the bone and from the bone back to the ultrasound probe can be measured. When a brain tissue is homogeneous, the edge of the bone is easy to detect both in the CT and in the ultrasound images. The speed of ultrasound waves S and the direction of the ultrasound waves from the initial propagation point P are shown in FIG. 2. If a shorter path between an initial point P and the bone structure 11 is possible, then the shorter path can be selected to determine the speed of the ultrasound waves (to be used where the output of the ultrasound apparatus is not sufficient or a low output is selected for medical reasons).

The tip P of the ultrasound probe and the orientation of the ultrasound probe can be determined from detecting the marker device using a navigation apparatus. The orientation of the ultrasound probe gives the direction of the speed S of the ultrasound waves and therefore also the direction relative to the bone structure. From this information, it is possible to determine the position of the reflective part of the bone structure. It is possible to determine the course of propagation of the ultrasound waves and the path length of the ultrasound waves from the initial location to the receiving location of the ultrasound waves. Scaled medical analysis data and/or medical analysis data (CT data) that is calibrated with respect to actual lengths may be used.

If the path length has been determined, then the speed of the ultrasound waves can be calculated from the path length using the transit time. A number of measurements may be taken to increase the accuracy and to compensate for registration errors (errors in determining the position of the ultrasound probe).

Alternatively or additionally, a number of ultrasound measurements (scans) can be used, wherein a number of courses (paths) of the ultrasound waves are detected by a single image. By correspondingly adapting (scaling) the ultrasound waves to the scaling of the medical data, it is possible to scale the ultrasound image and also to measure the speed of the ultrasound waves.

Ultrasound image scaling is based on the speed of sound. Some ultrasound devices allow a user to select a speed of sound to be used in scaling. Using these devices, it is possible (after the speed of sound has been measured) to directly set the corresponding result of the measurement on the device to obtain more accurate ultrasound images.

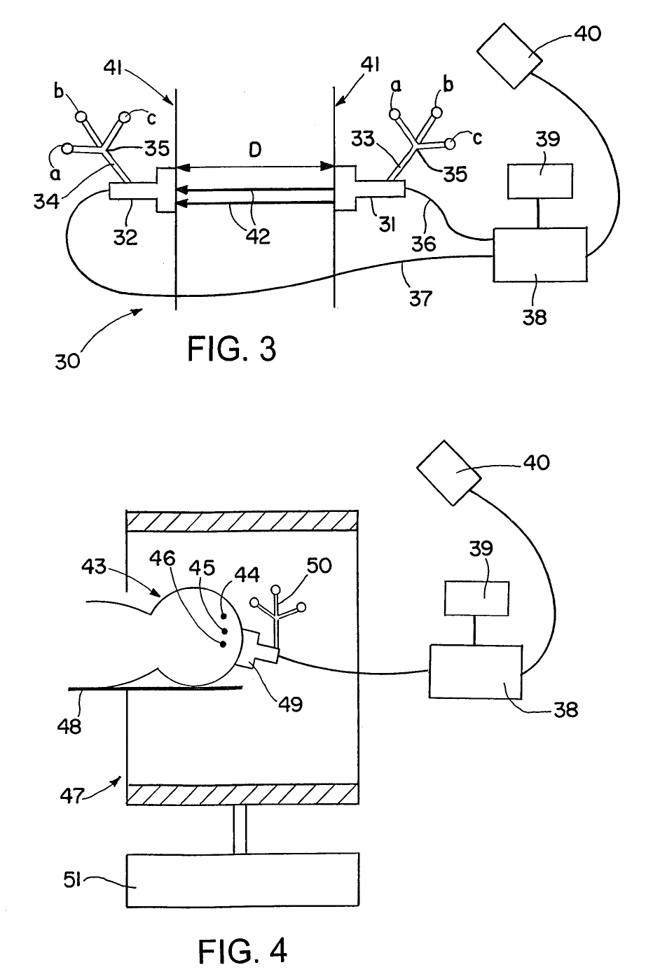

FIG. 3 schematically shows the speed of ultrasound being measured when using a two-part sonographic apparatus. The sonographic apparatus 30 includes a transmitter 31, a receiver 32, a transmitter marker device 33 and a receiver marker device 34. The marker devices 33 and 34 each include marker spheres a, b, c that are held in fixed positions relative to each other by a reference star 35. Control signals and detection signals from the ultrasound transmitter 31 and the ultrasound receiver 32 are transferred via lines 36 and 37 to an evaluation apparatus, such as a data processing device or computer 38. The data can be displayed on a monitor 39. A detection device, such as a camera 40, detects the positions of the marker devices 33 and 34. The detected positions are also transferred to the computer 38. The ultrasound propagates through a body structure 41, for example a leg, along the lines 42. D indicates the distance between the ultrasound transmitter 31 and the ultrasound receiver 32. The ultrasound waves require the transit time T to traverse the distance D. The speed of the ultrasound waves S is calculated from D divided by T, wherein the transit time T is calculated as the difference between the time of emitting an ultrasound pulse from the transmitter 31 and the time the ultrasound pulse is received by the receiver 32. The lines 36 and 37 transfer electrical control signals representing these pulses or other signals representing the emission/ reception times to the computer 38.

FIG. 4 illustrates an exemplary use of the method with respect to a patient's head 43. Marker spheres 44, 45, and 46 are implanted in the patient's head 43. The marker spheres (44, 45, 46) can be detected both optically by a camera 40 and by a CT apparatus (C-arc) 47. In the example, the patient is lying on a couch 48. The CT apparatus 47 takes X-ray recordings from different directions, to obtain a three-dimensional x-ray image. An ultrasound probe 49 including a marker device 50 is attached to the head of the patient, such that a fixed spatial relationship between the patient's head and the ultrasound probe results. The spacial relationship remains constant during measuring. The camera 40 ascertains the positions of the marker spheres (44, 45, 46) and the marker device 50. The position data is processed by the computer 38 and can be displayed on the monitor 39. The computer 38 also receives data from a data processing part 51 of the CT apparatus 47. Thus, by controlling the ultrasound probe 49 and the CT apparatus 47, the computer 38 can obtain all the data, in particular the reflection data, the location data, the marker data, and the transit time data, and use this data to calculate the speed of the ultrasound waves.

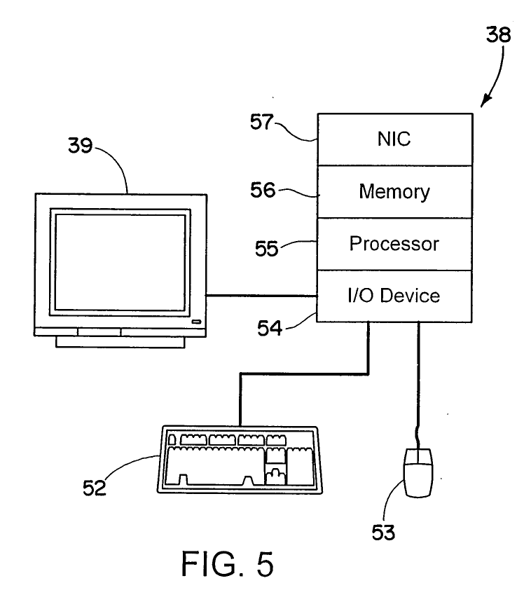

Moving now to FIG. 5 there is shown a block diagram of an exemplary computer 38 that may be used to implement one or more of the methods described herein. The computer 38 may be a standalone computer, or it may be part of a medical navigation system, for example. The computer 38 may include a display or monitor 39 for viewing system information, and a keyboard 52 and pointing device 53 for data entry, screen navigation, etc. Examples of a pointing device 53 include a computer mouse or other device that points to or otherwise identifies a location, action, etc., e.g., by a point and click method or some other method. Alternatively, a touch screen (not shown) may be used in place of the keyboard 52 and pointing device 53. The display 39, keyboard 52 and mouse 53 communicate with a processor via an input/output device 54, such as a video card and/or serial port (e.g., a USB port or the like).

A processor 55, such as an AMD Athlon 64® processor or an Intel Pentium IV® processor, combined with a memory 56 execute programs to perform various functions, such as data entry, numerical calculations, screen display, system setup, etc. The memory 56 may comprise several devices, including volatile and non-volatile memory components. Accordingly, the memory 56 may include, for example, random access memory (RAM), read-only memory (ROM), hard disks, floppy disks, optical disks (e.g., CDs and DVDs), tapes, flash devices and/or other memory components, plus associated drives, players and/or readers for the memory devices. The processor 55 and the memory 56 are coupled using a local interface (not shown). The local interface may be, for example, a data bus with accompanying control bus, a network, or other subsystem.

The memory may form part of a storage medium for storing information, such as application data, screen information, programs, etc., part of which may be in the form of a database. The storage medium may be a hard drive, for example, or any other storage means that can retain data, including other magnetic and/or optical storage devices. A network interface card (NIC) 57 allows the computer 38 to communicate with other devices.

A person having ordinary skill in the art of computer programming and applications of programming for computer systems would be able in view of the description provided herein to program a computer system 38 to operate and to carry out the functions described herein. Accordingly, details as to the specific programming code have been omitted for the sake of brevity. Also, while software in the memory 56 or in some other memory of the computer and/or server may be used to allow the system to carry out the functions and features described herein in accordance with the preferred embodiment of the invention, such functions and features also could be carried out via dedicated hardware, firmware, software, or combinations thereof, without departing from the scope of the invention.

Computer program elements of the invention may be embodied in hardware and/or in software (including firmware, resident software, micro-code, etc.). The invention may take the form of a computer program product, which can be embodied by a computer-usable or computer-readable storage medium having computer-usable or computer-readable program instructions, “code” or a “computer program” embodied in the medium for use by or in connection with the instruction execution system. In the context of this document, a computer-usable or computer-readable medium may be any medium that can contain, store, communicate, propagate, or transport the program for use by or in connection with the instruction execution system, apparatus, or device. The computer-usable or computer-readable medium may be, for example but not limited to, an electronic, magnetic, optical, electromagnetic, infrared, or semiconductor system, apparatus, device, or propagation medium such as the Internet. Note that the computer-usable or computer-readable medium could even be paper or another suitable medium, upon which the program is printed, as the program can be electronically captured, via, for instance, optical scanning of the paper or other medium, then compiled, interpreted, or otherwise processed in a suitable manner. The computer program product and any software and hardware described herein form the various means for carrying out the functions of the invention in the example embodiments.

Although the invention has been shown and described with respect to a certain preferred embodiment or embodiments, it is obvious that equivalent alterations and modifications will occur to others skilled in the art upon the reading and understanding of this specification and the annexed Figures. In particular regard to the various functions performed by the above described elements (components, assemblies, devices, software, computer programs, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for measuring a speed of ultrasound waves through a body structure, comprising:
providing data regarding a position of an ultrasound reflector relative to a first trackable marker;
providing data regarding a position of at least one of an ultrasound source and an ultrasound receiver relative to a second trackable marker;
determining a path length traveled by the ultrasound waves from the ultrasound source to the ultrasound reflector and from the ultrasound reflector to the ultrasound receiver based on a position of the first trackable maker relative to the second trackable marker;
measuring a transit time for the ultrasound waves to travel from the ultrasound source to the ultrasound receiver; and
calculating the speed of the ultrasound waves from the measured transit time and the determined path length.

2. The method according to claim 1, wherein the ultrasound source and the ultrasound receiver are in the same location.

3. The method according to claim 2, wherein the ultrasound source and the ultrasound receiver components of an ultrasound probe.

4. The method according to claim 3, wherein providing data regarding a position of at least one of an ultrasound source and an ultrasound receiver relative to a second trackable marker includes ultrasound probe calibration.

5. The method according to claim 1, wherein the ultrasound source has a fixed, predetermined positional relationship relative to the ultrasound receiver.

6. The method according to claim 1, wherein the ultrasound reflector is a part of the body structure that reflects the ultrasound waves.

7. The method according to claim 6, wherein the data regarding a position of an ultrasound reflector relative to a first trackable marker is obtained by an analysis method that does not use sonography.

8. The method according to claim 7, wherein providing data regarding a position of an ultrasound reflector relative to a first trackable marker includes patient registration.

9. The method according to claim 1, wherein the ultrasound reflector is situated outside a patient's body.

10. The method according to claim 1, further comprising scaling an ultrasound image using the calculated speed of the ultrasound waves.

11. The method according to claim 1, wherein a number of calculated speeds of ultrasound waves are averaged.

12. The method according to claim 1, further comprising:
providing body structure data identifying the position and type of different regions within the body structure,
providing speed-of-sound data regarding assignment of particular speed-of-sound values to each type of different region identified within the body structure,
assigning speed-of-sound values to each different region of the body structure based on the body structure data and the speed-of-sound data,
estimating a speed of ultrasound waves through the body structure based on the body structure data and the assigned speed-of-sound values; and
adjusting the assigned speed-of-sound values based on a comparison of the estimated speed of ultrasound waves to the calculated speed of the ultrasound waves.

13. The method according to claim 12, further comprising scaling at least a one part of an ultrasound image using the adjusted speed-of-sound values.

14. A method for measuring a speed of ultrasound waves through a body structure, comprising:
providing data regarding a position of an ultrasound source relative to a first trackable marker;
providing data regarding a position of an ultrasound receiver relative to a second trackable marker;
determining a path length traveled by the ultrasound waves from the ultrasound source to the ultrasound receiver based on a position of the first trackable maker relative to the second trackable marker;
measuring a transit time for the ultrasound waves to travel from the ultrasound source to the ultrasound receiver; and
calculating the speed of the ultrasound waves from the measured transit time and the determined path length.

15. A device for measuring a speed of ultrasound waves through a body structure, comprising:
a first trackable marker secured to an ultrasound reflector;
at least one of an ultrasound source and an ultrasound receiver;
a second trackable marker secured to the at least one of an ultrasound source and an ultrasound receiver;
a marker detection device for determining the positions of the first trackable marker and the second trackable marker; and
a computer operatively coupled to said marker detection device, said ultrasound source, and said ultrasound receiver, said computer comprising:
a processor and memory, and
logic stored in the memory and executable by the processor, said logic including:
i) logic that determines a path length traveled by the ultrasound waves from the ultrasound source to the ultrasound reflector and from the ultrasound reflector to the ultrasound receiver based on a position of the first trackable maker relative to the second trackable marker;
ii) logic that measures a transit time for the ultrasound waves to travel from the ultrasound source to the ultrasound receiver; and
iii) logic that calculates the speed of the ultrasound waves from the measured transit time and the determined path length.

16. A device for measuring a speed of ultrasound waves through a body structure, comprising:
an ultrasound source including a first trackable marker;
an ultrasound receiver including a second trackable marker;
a marker detection device for determining the positions of the first trackable marker and the second trackable marker; and
a computer operatively coupled to said marker detection device, said ultrasound source, and said ultrasound receiver, said computer comprising:
a processor and memory, and
logic stored in the memory and executable by the processor, said logic including:
i) logic that determines a path length traveled by the ultrasound waves from the ultrasound source to the ultrasound receiver based on a position of the first trackable maker relative to the second trackable marker;
ii) logic that measures a transit time for the ultrasound waves to travel from the ultrasound source to the ultrasound receiver; and
iii) logic that calculates the speed of the ultrasound waves from the measured transit time and the determined path length.

17. A computer program embodied on a computer readable medium for measuring the speed of ultrasound waves through a body structure, comprising:
a) code that determines the positions of an ultrasonic source, an ultrasonic receiver, and a reflection part in a reference coordinate system;
b) code that determines a path length for ultrasound waves to travel from the ultrasonic source to the ultrasonic receiver via reflection off the reflection part;
c) code that measures a transit time for the ultrasound waves to traverse the path length; and
d) code that determines the speed of ultrasound waves travelling from the ultrasonic source to the ultrasonic receiver based on the path length and the transit time.
